(19) United States
(12) Patent Application Publication
(10) Pub. No.: US 2007/0293474 A1
(43) Pub. Date: Dec. 20, 2007

(54) Combination of a DPP IV inhibitor and a cardiovascular compound

(76) Inventors: David Grenville Holmes, Binningen (CH); Suraj Shivappa Shetty, Far Hills, NJ (US); Thomas Edward Hughes, Concord, MA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/815,536

(22) Filed: Aug. 3, 2007

Related U.S. Application Data
(63) Continuation of application No. 10/515,864, filed on Dec. 21, 2004, now abandoned.

(30) Foreign Application Priority Data
May 29, 2002 (GB) 0212112.1

(51) Int. Cl. A61K 31/4439; A61K 31/135; A61K 31/40; A61K 31/403; A61K 31/496; A61K 31/549; A61P 17/00; A61P 3/00; A61P 3/10; A61P 9/12; A61P 3/04; A61P 27/06; A61K 31/55; A61K 31/513; A61K 31/407; A61K 31/41; A61K 31/44

(52) U.S. Cl. 514/212.07; 514/223.2; 514/252.17; 514/269; 514/343; 514/356; 514/381; 514/412; 514/423; 514/462; 514/651; 514/789

(57) ABSTRACT

The present invention relates to a combination, such as a combined preparation or pharmaceutical composition, respectively, comprising of a DPP IV inhibitor or a pharmaceutically acceptable salt thereof and a cardiovascular compound (being different from statin) or a pharmaceutically acceptable salt thereof. The present invention furthermore relates to the use of such a combination for the prevention, delay of progression or treatment of diseases and disorders.

The present invention relates to a combination, such as a combined preparation or pharmaceutical composition, respectively, comprising of a DPP IV inhibitor or a pharmaceutically acceptable salt thereof and a cardiovascular compound (being different from a statin) or a pharmaceutically acceptable salt thereof.

The invention especially relates to a combination, such as a combined preparation or pharmaceutical composition, respectively, comprising a DPP IV inhibitor or a pharmaceutically acceptable salt thereof and at least one cardiovascular compound, i.e. a therapeutic agent selected from the group consisting of

(i) an AT₁-receptor antagonist or a pharmaceutically acceptable salt thereof,

(ii) an angiotensin converting enzyme (ACE) inhibitor or a pharmaceutically acceptable salt thereof,

(iii) a renin inhibitor or a pharmaceutically acceptable salt thereof,

(iv) a beta adrenergic receptor blocker or a pharmaceutically acceptable salt thereof,

(v) an alpha adrenergic receptor blocker or a pharmaceutically acceptable salt thereof,

(vi) a calcium channel blocker or a pharmaceutically acceptable salt thereof,

(vii) an aldosterone synthase inhibitor or a pharmaceutically acceptable salt thereof,

(viii) an aldosterone receptor antagonist or a pharmaceutically acceptable salt thereof,

(ix) a neutral endopeptidase (NEP) inhibitor or a pharmaceutically acceptable salt thereof,

(x) a dual angiotensin converting enzyme/neutral endopeptidase (ACE/NEP) inhibitor or a pharmaceutically acceptable salt thereof,

(xi) an endothelin receptor antagonist or a pharmaceutically acceptable salt thereof,

(xii) a diuretic or a pharmaceutically acceptable salt thereof.

The term “at least one therapeutic agent” shall mean that in addition to the compound of formula (I) one or more, for example two, furthermore three, active ingredients as specified according to the present invention can be combined.

The term “DPP-IV” as used herein is intended to mean dipeptidyl peptidase IV, also known as CD26. DPP-IV, a serine protease belonging to the group of post proline/alanine cleaving amino-dipeptidases, specifically removes the two N-terminal amino acids from proteins having proline or alanine in position 2. DPP-IV can be used in the control of glucose metabolism because its substrates include the insulinotropic hormones glucagon like peptide-1 (GLP-1) and gastric inhibitory peptide (GIP). GLP-1 and GIP are active only in their intact forms; removal of their two N-terminal amino acids inactivates them.

In vivo administration of synthetic inhibitors of DPP-IV prevents N-terminal degradation of GLP-1 and GIP, resulting in higher plasma concentrations of these hormones, increased insulin secretion and, therefore, improved glucose tolerance.

The term “DPP-IV inhibitor” is intended to indicate a molecule that exhibits inhibition of the enzymatic activity of DPP-IV and functionally related enzymes, such as from 1-100% inhibition, and specially preserves the action of substrate molecules, including but not limited to GLP-1, GIP, peptide histidine methionine, substance P, neuropeptide Y, and other molecules typically containing alanine or proline residues in the second amino terminal position. Treatment with DPP-IV inhibitors prolongs the duration of action of peptide substrates and increases levels of their intact, undegraded forms leading to a spectrum of biological activities relevant to the disclosed invention.

For that purpose, chemical compounds are tested for their ability to inhibit the enzyme activity of purified CD26/DPP-IV. Briefly, the activity of CD26/DPP-IV is measured in vitro by its ability to cleave the synthetic substrate Gly-Pro-p-nitroanilide (Gly-Pro-pNA). Cleavage of Gly-Pro-pNA by DPP-IV liberates the product p-nitroanilide (pNA), whose rate of appearance is directly proportional to the enzyme activity. Inhibition of the enzyme activity by specific enzyme inhibitors slows down the generation of pNA. Stronger interaction between an inhibitor and the enzyme results in a slower rate of generation of pNA. Thus, the degree of inhibition of the rate of accumulation of pNA is a direct measure of the strength of enzyme inhibition. The accumulation of pNA is measured spectrophotometrically. The inhibition constant, Ki, for each compound is determined by incubating fixed amounts of enzyme with several different concentrations of inhibitor and substrate.

In the present context “a DPP-IV inhibitor” is also intended to comprise active metabolites and prodrugs thereof, such as active metabolites and prodrugs of DPP-IV inhibitors. An active “metabolite” is an active derivative of a DPP-IV inhibitor produced when the DPP-IV inhibitor is metabolized. A “prodrug” is a compound that is either metabolized to a DPP-IV inhibitor or is metabolized to the same metabolite(s) as a DPP-IV inhibitor.

DPP-IV inhibitors are known in the art. For example, DPP-IV inhibitors are in each case generically and specifically disclosed e.g. in WO 98/19998, DE19616 486 A1, WO 00/34241, WO 95/15309, WO 01/72290, WO01/52825, WO 9310127, WO 9925719, WO 9938501, WO 9946272, WO 9967278 and WO 9967279. In each case in particular in the compound claims and the final products of the working examples, the subject matter of the final products, the pharmaceutical preparations and the claims are hereby incorporated into the present application by reference to these publications.

Published patent application WO 9819998 discloses N—(N′-substituted glycyl)-2-cyano pyrrolidines, in particular 1-[2-[5-Cyanopyridin-2-yl]amino]-ethylamino]acetyl-2-cyano-(S)-pyrrolidine (NVP-DPP728).

DE19616 486 A1 discloses val-pyr, val-thiazolidide, isoleucyl-thiazolidide, isoleucyl-pyrrolidide, and fumar salts of isoleucyl-thiazolidide and isoleucyl-pyrrolidide.

Published patent application WO 0034241 and published patent U.S. Pat. No. 6,110,949 disclose N-substituted adamantyl-amino-acetyl-2-cyano pyrrolidines and W (substituted glycyl)-4-cyano pyrrolidines respectively. DPP-IV inhibitors of interest are specially those cited in claims 1 to 4.

Published patent application WO 9515309 discloses amino acid 2-cyanopyrrolidine amides as inhibitors of DPP-IV Published patent application WO 9529691 discloses peptidyl derivates of diesters of alpha-aminoalkylphosphonic acids, particularly those with proline or related structures. DPP-IV inhibitors of interest are specially those cited in Table 1 to 8.

In WO 01/72290 DPP-IV inhibitors of interest are specially those cited in example 1 and claims 1, 4, and 6.

WO01/52825 specially discloses (S)-1-{2-[5-cyanopyridin-2-yl)amino]ethyl-aminoacetyl)-2-cyano pyrrolidine or (S)-1-[(3-hydroxy-1-adamantyt)amino]acetyl-2-cyano-pyrrolidine.

Published patent application WO 9310127 discloses proline boronic esters useful as DPP-IV inhibitors. DPP-IV inhibitors of interest are specially those cited in examples 1 to 19.

Published patent application WO 9925719 discloses sulphostin, a DPP-IV inhibitor prepared by culturing a Streptomyces microorganism.

Published patent application WO 9938501 discloses N-substituted 4-8 membered heterocyclic rings. DPP-IV inhibitors of interest are specially those cited in claims 15 to 20

Published patent application WO 9946272 discloses phosphoric compounds as inhibitors of DPP-IV. DPP-IV inhibitors of interest are specially those cited in claims 1 to 23.

Published patent applications WO 9967278 and WO 9967279 disclose DPP-IV prodrugs and inhibitors of the form A-B-C where C is either a stable or unstable inhibitor of DPP-IV. Any of the substances disclosed in the above mentioned patent documents, hereby included by reference, are considered potentially useful as DPP-IV inhibitors to be used in carrying out the present invention.

Preferred DPP-IV inhibitors are N-substituted adamantyl-amino acetyl-2-cyano pyrrolidines, N (substituted glycyl)-4-cyano pyrrolidines, N—(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl)amino]ethylamino]acetyl-2-cyano-(S)-pyrrolidine and pharmaceutical salts thereof.

Especially preferred are 1-{2-[(5-cyanopyridin-2-yl)amino]ethylamino}acetyl-2 (S)-cyano-pyrrolidine dihydrochloride, of formula 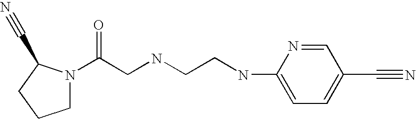
especially the dihydrochloride thereof,
and pyrrolidine, 1-[(3-hydroxy-1-adamantyl)amino]acetyl-2-cyano-, (S) of formula 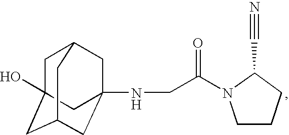
L-threo-isoleucyl thiazolidine (compound code according to Probiodrug: P32198), and pharmaceutical salts thereof.

Especially preferred are orally active DPP-IV inhibitors.

AT₁-receptor antagonists (also called angiotensin II receptor antagonists or angiotensin II receptor blockers) are understood to be those active ingredients that bind to the AT₁-receptor subtype of angiotensin II receptor but do not result in activation of the receptor. As a consequence of the inhibition of the AT₁ receptor, these antagonists can, for example, be employed as antihypertensives or for treating congestive heart failure.

The class of AT₁ receptor antagonists comprises compounds having differing structural features, essentially preferred are the non-peptidic ones. For example, mention may be made of the compounds that are selected from the group consisting of valsartan (of. EP 443983), losartan (cf. EP253310), candesartan (cf. 459136), eprosartan (cf. EP 403159), irbesartan (cf. EP454511), olmesartan (cf. EP 503785), tasosartan (cf. EPS39086), telmisartan (of. EP 522314), saprisartan, the compound with the designation E-1477 of the following formula 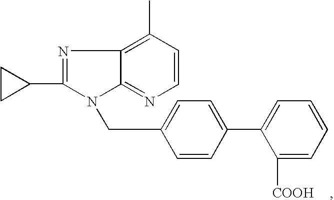
the compound with the designation SC-52458 of the following formula 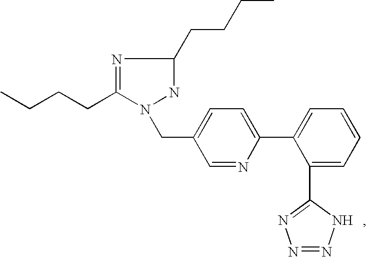
and the compound with the designation the compound ZD-8731 of the following formula 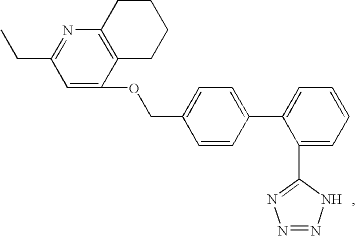
or, in each case, a pharmaceutically acceptable salt thereof.

Preferred AT₁-receptor antagonist are those agents that have been marketed, most preferred is valsartan or a pharmaceutically acceptable salt thereof.

The interruption of the enzymatic degradation of angiotensin I to angiotensin II with so-called AGE-inhibitors (also called angiotensin converting enzyme inhibitors) is a successful variant for the regulation of blood pressure and also a therapeutic method for the treatment of congestive heart failure.

The class of ACE inhibitors comprises compounds having differing structural features. For example, mention may be made of the compounds which are selected from the group consisting alacepril, benazepril, benazeprilat, captopril, ceronapril, cilazapril, delapril, enalapril, enalaprilat, fosinopril, imidapril, lisinopril, moexipril, moveltopril, pentopril, perindopril, quinapril, quinaprilat, ramipril, ramiprilat, spirapril, temocapril, trandolapril and zofenopril, or, in each case, a pharmaceutically acceptable salt thereof.

Preferred ACE inhibitors are those agents that have been marketed, most preferred are benazepril, ramipril, lisinopril and enalapril.

Renin released from the kidneys cleaves angiotensinogen in the circulation to form the decapeptide angiotensin I. This is in turn cleaved by angiotensin converting enzyme in the lungs, kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume. Inhibitors of the enzymatic activity of renin bring about a reduction in the formation of angiotensin I. As a result a smaller amount of angiotensin II is produced. The reduced concentration of that active peptide hormone is the direct cause of e.g. the antihypertensive effect of renin inhibitors. Accordingly, renin inhibitors or salts thereof can be employed e.g. as antihypertensives or for treating congestive heart failure.

The class of renin inhibitors comprises compounds having differing structural features. For example, mention may be made of compounds which are selected from the group consisting of ditekiren (chemical name: [1S-[1R*,2R*,4R*(1R*,2R*)]]-1-[(1,1-dimethylethoxy)carbonyl]-L-prolyl-L-phenylalanyl-N-[2-hydroxy-5-methyl-1-(2-methylpropyl)-4-[[[2-methyl-1-[[{(2-pyridinylmrthyl)amino]carbonyl]butyl]amino}carbonyl]hexyl]-N-alfa-methyl-L-histidinamide); terlakiren (chemical name: [R—(R*,S*)]-N-(4-morpholinylcarbonyl)-L-phenylalanyl-N-[1-(cyclohexylmethyl)-2-hydroxy-3-(1-methylethoxy)-3-oxopropyl]-S-methyl-L-cysteineamide); zankiren (chemical name: 1S-[1R*[R*(R*)],2S*,3R*]]-N-[1-(cyclohexylmethyl)-2,3-dihydroxy-5-methylhexyl]-alfa-[[2-[[(4-methyl-1-piperazinyl)sulfonyl]methyl]-1-oxo-3-phenylpropyl]amino]-4-thiazolepropanamide), especially the hydrochloride thereof; RO 66-1132 and RO-66-1168 of formulae

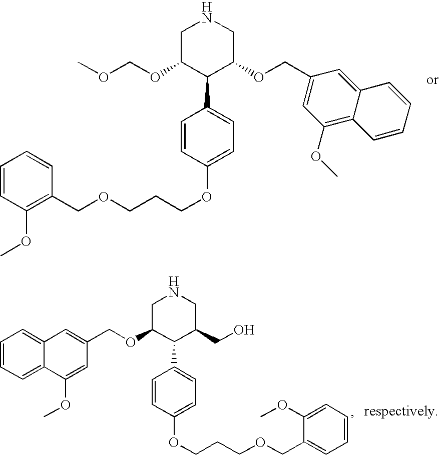

Especially preferred is the compound of formula 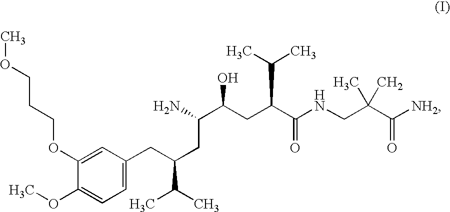
chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide (generic name: aliskiren), specifically disclosed in EP 678503 A, or a pharmaceutically acceptable salt, especially the hemi-fumarate, thereof.

A beta adrenergic receptor blocker in said combination preferably is a representative selected from the group consisting of a selective β1-blocker, such as atenolol, bisoprolol (especially the fumarate thereof), metoprolol (especially the hemi-(R,R)fumarate or fumarate thereof), esmolol (especially the hydrochloride thereof, celiprolol (especially the hydrochloride thereof), betaxolol (especially the hydrochloride thereof) or taliprolol, or, a non-selective β-blocker, such as oxprenolol (especially the hydrochloride thereof), pindolol, propranolol (especially the hydrochloride thereof), timolol (especially the maleate thereof), bupranolol (especially the hydrochloride thereof), penbutolol (especially the sulphate thereof), mepindolol (especially the sulphate thereof), carteolol (especially the hydrochloride thereof) or nadolol, and a β-blocker with α-blocking activity such as carvedilol or labetalol; or in each case, a pharmaceutically acceptable salt thereof.

An alpha₁ adrenergic receptor blocker in said combination preferably is a representative selected from the group consisting of doxazosin, prazosin or terazosin; or in each case, a pharmaceutically acceptable salt thereof. All of these alpha₁ adrenergic receptor blockers are used as antihypertensive drugs.

The class of calcium channel blockers (CCBs) essentially comprises dihydropyridines (DHPs) and non-DHPs such as diltiazem-type and verapamil-type CCBs. A CCB useful in said combination is preferably a DHP representative selected from the group consisting of amlodipine, felodipine, ryosidine, isradipine, lacidipine, nicardipine, nifedipine, niguldipine, niludipine, nimodipine, nisoldipine, nitrendipine, and nivaldipine, and is preferably a non-DHP representative selected from the group consisting of flunarizine, prenylamine, diltiazem, fendiline, gallopamit, mibefradil, anipamil, tiapamil and verapamil, and in each case, a pharmaceutically acceptable salt thereof. All these CCBs are therapeutically used, e.g. as anti-hypertensive, anti-angina pectoris or anti-arrhythmic drugs.

Preferred CCBs comprise amlodipine, diltiazem, isradipine, nicardipine, nifedipine, nimodipine, nisoldipine, nitrendipine, and verapamil, or, e.g. dependent on the specific CCB, a pharmaceutically acceptable salt thereof. An especially preferred DHP is amlodipine or a pharmaceutically acceptable salt, especially the besylate, thereof. An especially preferred representative of non-DHPs is verapamil or a pharmaceutically acceptable salt, especially the hydrochloride, thereof.

Aldosterone synthase is an enzyme that converts corticosterone to aldosterone by hydroxylating corticosterone to form 18-OH-corticosterone and 18-OH-corticosterone to aldosterone. The class of aldosterone synthase inhibitors is known to be applied for the treatment of hypertension and primary aldosteronism comprises both steroidal and non-steroidal aldosterone synthase inhibitors, the later being most preferred.

Preference is given to commercially available aldosterone synthase inhibitors or those aldosterone synthase inhibitors that have been approved by the health authorities.

The class of aldosterone synthase inhibitors comprises compounds having differing structural features. For example, mention may be made of the compounds which are selected from the group consisting of the non-steroidal aromatase inhibitors anastrozole, fadrozole (including the (+)-enantiomer thereof), as well as the steroidal aromatase inhibitor exemestane, or, in each case where applicable, a pharmaceutically acceptable salt thereof.

The most preferred non-steroidal aldosterone synthase inhibitor is the (+)-enantiomer of the hydrochloride of fadrozole (U.S. Pat. Nos. 4,617,307 and 4,889,861) of formula 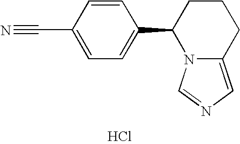
or a pharmaceutically acceptable alternate salt form thereof.

A preferred steroidal aldosterone receptor antagonist is eplerenone (cf. EP 122232 A) of the formula 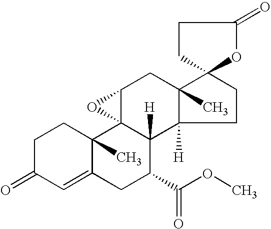
or spironolactone.

The natriuretic peptides constitute a family of peptides that include the atrial (ANP), brain-derived (BNP) and C-type natriuretic (CNP) peptides. The natriuretic peptides effect vasodilation, natriuresis, diuresis, decreased aldosterone release, decreased cell growth, and inhibition of the sympathetic nervous system and the renin-angiotensin-aldosterone system indicating their involvement in the regulation of blood pressure and of sodium and water balance. Neutral endopeptidase 24.11 (NEP) inhibitors impede degradation of natriuretic peptides and elicit pharmacological actions potentially beneficial in the management of several cardiovascular disorders. A NEP inhibitor useful in the said combination is an agent selected from the group represented by candoxatril, sinorphan, SCH 34826 and SCH 42495.

Compounds having inhibitory effects on both angiotensin converting enzyme and neutral endopetidase, so-called dual ACE/NEP inhibitors, can be used for the treatment of cardiovascular pathologies. A preferred dual angiotensin converting enzyme/neutral endopetidase (ACE/NEP) inhibitor is, for example, omapatrilat (of. EP 629627), fasidotril or fasidotrilat (of. EP 419327), or Z 13752A (cf. WO 97/24342) or, if appropriate, a pharmaceutically acceptable salt thereof.

Endothelin (ET) is a highly potent vasoconstrictor peptide synthesized and released by the vascular endothelium. Endothelin (ET) exists in three isoforms (ET-1, ET-2 and ET-3). (ET shall mean any or all of the isoforms of ET). Elevated levels of ET have been reported in plasma from patients with e.g. essential hypertension. Endothelin receptor antagonists can be used to inhibit the vasoconstrictive effects induced by ET.

A preferred endothelin antagonist is, for example, bosentan (cf. EP 526708 A), enrasentan (cf. WO 94/25013), atrasentan (cf. WO 96/06095), especially atrasentan hydrochloride, darusentan (cf. EP 785926 A), BMS 193884 (cf. EP 702012 A), sitaxsentan (cf. U.S. Pat. No. 5,594,021), especially sitaxsentan sodium, YM 598 (cf. EP 882719 A), S 0139 (cf. WO 97/27314), J 104132 (cf. EP 714897 A or WO 97/37665), furthermore, tezosentan (cf. WO 96/19459), or in each case, a pharmaceutically acceptable salt thereof.

A diuretic is, for example, a thiazide derivative selected from the group consisting of chlorothiazide, hydrochlorothiazide, methylchlorothiazide, and chlorothalidon. The most preferred is hydrochlorothiazide.

Preferred are combinations, such as combined preparations or pharmaceutical compositions, respectively, comprising the DPP-IV inhibitor of formula (I) or a pharmaceutically accepted salt thereof and as second active agent an active agent selected from the group consisting of valsartan, benazepril, ramipril, lisinopril, enalapril, amlodipine, especially the besylate thereof, aliskiren, especially the hemifumarate thereof, atenolol, metoprolol, especially the hemi (R,R)fumarate or the fumarate thereof, oxprenolol, doxazosin, the (+) enantiomer of fadrozole, eplerenone, omapatrilat, Z 13752A, sitaxsentan, especially sitaxsentan sodium, darusentan and hydrochlorothiazide.

Furthermore preferred are combinations, such as a combined preparations or pharmaceutical compositions, respectively, comprising the DPP-IV inhibitor of formula (I) or a pharmaceutically accepted salt thereof and one active agent selected from the group consisting of valsartan, benazepril, ramipril, lisinopril, enalapril, amlodipine, especially the besylate thereof, aliskiren, especially the hemifumarate thereof, atenolol, metoprolol, especially the hemi (R,R)fumarate or the fumarate thereof, oxprenolol, doxazosin, the (+) enantiomer of fadrozole, eplerenone, omapatrilat, Z 13752A, sitaxsentan, especially sitaxsentan sodium, and darusentan, furthermore comprising as third active agent hydrochlorothiazide.

The structure of the active agents identified by generic or tradenames may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

The corresponding active ingredients or a pharmaceutically acceptable salts thereof may also be used in form of a solvate, such as a hydrate or including other solvents, used for crystallization.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases.

All the more surprising is the experimental finding that the combined administration of a DPP IV inhibitor or a salt thereof and a therapeutic agent selected from the group consisting of (i) to (xii) results not only in a beneficial, especially a synergistic, therapeutic effect, but also in additional benefits resulting from the combined treatment and further surprising beneficial effects compared to a monotherapy applying only one of the pharmaceutically active compounds used in the combinations disclosed herein.

It can be shown by established test models and especially those test models described herein that the combination of the DPP-IV inhibitor of formula (I) with a therapeutic agent selected from the group consisting of (i) to (xii) results in a more effective prevention or preferably treatment of diseases specified in the following. In particular, it can be shown by established test models and especially those test models described herein that the combination of the present invention results in a more effective prevention or preferably treatment of diseases specified hereinafter.

If taken simultaneously, this results not only in a further enhanced beneficial, especially a synergistic, therapeutic effect, but also in additional benefits resulting from the simultaneous treatment such as a surprising prolongation of efficacy, a broader variety of therapeutic treatment and surprising beneficial effects, e.g. less increase of weight, on diseases and conditions associated with diabetes mellitus, for a number of combinations as described herein. Moreover, for a human patient, especially for elderly people, it is more convenient and easier to remember to take two tablets at the same time, e.g. before a meal, than staggered in time, i.e. according to a more complicated treatment schedule. More preferably, both active ingredients are administered as a fixed combination, i.e. as a single tablet, in all cases described herein. Taking a single tablet is even easier to handle than taking two tablets at the same time. Furthermore, the packaging can be accomplished with less effort.

The person skilled in the pertinent art is fully enabled to select a relevant and standard animal test model to prove the hereinbefore and hereinafter indicated therapeutic indications and beneficial effects.

The pharmaceutical activities as effected by administration of the DPP-IV inhibitor of formula (I) or of the combination of the active agents used according to the present invention can be demonstrated e.g. by using corresponding pharmacological models known in the pertinent art.

To evaluate the antihypertensive activity of the combination according to the invention, for example, the methodology as described by Lovenberg W: Animal models for hypertension research Prog. Clin. Biol. Res. 1987, 229, 225-240 may be applied. For the evaluation that the combination according to the present invention may be used for the treatment of congestive heart failure, for example, the methods as disclosed by Smith H J, Nuttall A: Experimental models of heart failure. Cardiovasc Res 1985, 19, 181-186 may be applied. Also, rat models of hypertension and cardiac failure as described by Doggrell S A and Brown L. (Cardiovasc Res 1998, 39: 89-105) may be used for the pharmacological evaluation of the combination. Molecular approaches such as transgenic methods are also described, for example by Luft et al.: Hypertension-induced end-organ damage. “A new transgenic approach for an old problem.” Hypertension 1999, 33, 212-218.

The insulin secretion enhancing properties of the combination according to the present invention may be determined by following the methodology as disclosed, for example, in the publication of T. Ikenoue et al. Biol. Pharm. Bull. 29(4), 354-359 (1997).

The simultaneous evaluation of the cardiovascular actions and of the glucose utilization effects of the agents given alone or in combination can be performed using models such as the Zucker fatty rat as described in the publication of Nawano et al., Metabolism 48: 1248-1255, 1999. Also, studies using diabetic spontaneously hypertensive rats are described in the publication of Sato et al., Metabolism 45:457-462, 1996. Furthermore, rat models such as the Cohen-Rosenthal diabetic hypertensive rat (Rosenthal et al., Hypertension. 1997; 29:1260-1264) may also be used for the simultaneous assessments of the effects of the combination on blood pressure and glucose metabolism.

The corresponding subject matter of these eight references is herewith incorporated by reference in this specification.

Accordingly, the combination according to the present invention may be used, e.g., for the prevention, delay of progression or treatment of diseases and disorders that may be inhibited by DPP IV inhibition, that may be inhibited by the enhancement of insulin secretion and that may be inhibited by insulin sensitization. Especially, the combination according to the present invention may be used, e.g., for the prevention, delay of progression or treatment of diseases and disorders selected from the group consisting of hypertension (including but not limited to isolated systolic hypertension and familial dyslipidemic hypertension), congestive heart failure, left ventricular hypertrophy, peripheral arterial disease, diabetes, especially type 2 diabetes mellitus, diabetic retinopathy, macular degeneration, cataract, diabetic nephropathy, glomerulosclerosis, chronic renal failure, diabetic neuropathy, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertryglyceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance. Preferably, said combination may be used for the treatment of hypertension, especially isolated systolic hypertension (ISH), congestive heart failure, endothelial dysfunction, impaired vascular compliance, impaired glucose tolerance and type II diabetes mellitus.

A “disease or condition which may be inhibited by a DPP-IV inhibitor” as defined in this application comprises, but is not limited to insulin resistance, impaired glucose metabolism, conditions of impaired glucose tolerance, conditions of impaired fasting plasma glucose, obesity, diabetic retinopathy, macular degeneration, cataracts, diabetic nephropathy, glomerulosclerosis, diabetic neuropathy, erectile dysfunction, premenstrual syndrome, coronary heart disease, hypertension, angina pectoris, myocardial infarction, stroke, vascular restenosis, skin and connective tissue disorders, foot ulcerations and ulcerative colitis, endothelial dysfunction and impaired vascular compliance.

Hypertension, in connection with a “disease or condition which may be inhibited by a cardiovascular compound [selected from the group (i)-(xii)]”, a “disease or condition which may be inhibited by the enhancement of insulin secretion” includes and is not limited to mild, moderate and severe hypertension as defined in Journal of Hypertension 1999, 17:161-183, especially on page 162. Especially preferred is ISH. ISH is the most common form of hypertension in people over 50 years. It is defined as elevated systolic blood pressure (above 140 mm Hg) in conjunction with normal diastolic blood pressure (below 90 mm Hg). Elevated systolic blood pressure is an independent risk factor for cardiovascular diseases and may lead e.g. to myocardial hypertrophy and heart failure. ISH is furthermore characterized by an increased pulse pressure, defined as the difference between systolic and diastolic blood pressures. Elevated pulse pressure is being recognized as the type of hypertension the least likely to be well controlled. A reduction of elevated systolic blood pressure and correspondingly of pulse pressure is associated with a significant risk reduction in cardiovascular death. It has surprisingly been found that the combination of a DPP-IV inhibitor and a cardiovascular compound, as described in the present invention, leads to a decrease of ISH and pulse rate, both in hypertensive patients having type 2 diabetes mellitus and in hypertensive patients that do not have type 2 diabetes mellitus.

The term “prevention” means prophylactic administration of the combination to healthy patients to prevent the outbreak of the conditions mentioned herein. Moreover, the term “prevention” means prophylactic administration of such combination to patients being in a pre-stage of the conditions, to be treated.

The term “delay of progression” used herein means administration of the combination, such as a combined preparation or pharmaceutical composition, to patients being in a pre-stage of the condition to be treated in which patients a pre-form of the corresponding condition is diagnosed. Included is ‘prehypertension’ with ‘compelling indications’ as defined in the JNC 7 Report (JAMA 2003, 289:2560-2572). Prehypertension is defined as systolic blood pressure ranging from 120-139 mm Hg or diastolic blood pressure ranging from 80-89 mm Hg.

By the term “treatment” is understood the management and care of a patient for the purpose of combating the disease, condition, or disorder.

Preferably, the jointly therapeutically effective amounts of the active agents according to the combination of the present invention can be administered simultaneously or sequentially in any order, e.g. separately or in a fixed combination.

Under certain circumstances, drugs with different mechanisms of action may be combined. However, just considering any combination of drugs having different modes of action but acting in the similar field does not necessarily lead to combinations with advantageous effects.

All the more surprising is the experimental finding that the combined administration of a DPP-IV inhibitor according to the present invention, or, in each case, a pharmaceutically acceptable form thereof, results not only in a beneficial, especially a potentiating or a synergistic, therapeutic effect. Independent thereof, additional benefits resulting from combined treatment can be achieved such as a surprising prolongation of efficacy, a broader variety of therapeutic treatment and surprising beneficial effects on diseases and conditions associated with diabetes, e.g. less gain of weight. An additional and preferred aspect of the present invention is the prevention, delay of progression or treatment of the condition of isolated systolic hypertension and impaired vascular compliance which means decreased vascular elasticity.

The term “potentiation” shall mean an increase of a corresponding pharmacological activity or therapeutical effect, respectively. Potentiation of one component of the combination according to the present invention by co-administration of another component according to the present invention means that an effect is being achieved that is greater than that achieved with one component alone.

The term “synergistic” shall mean that the drugs, when taken together, produce a total joint effect that is greater than the sum of the effects of each drug when taken alone.

The diseases, disorders or conditions related to type 2 diabetes mellitus, includes but are not limited to diabetic nephropathy, diabetic retinopathy and diabetic neuropathy.

Furthermore, it has been found that the chronic co-administration of either an insulin sensitizer or an insulin secretion enhancer imparts the beneficial effect on blood vessel morphology and function and results in a decrease of vascular stiffness and correspondingly in a maintenance and in an improvement of vascular compliance.

Accordingly, it has been found that the addition of a DPP-IV inhibitor to that of a cardiovascular compound would potentiate the effect on systolic blood pressure and further improve vascular stiffness/compliance. Conversely, the proven antihypertensive effects of a cardiovascular compound on systolic and diastolic blood pressure may be potentiated by the addition of a DPP-IV inhibitor. The benefit of these combinations may also extend to an additional or potentiated effect on endothelial function, and improve vascular function and structure in various organs/tissues including the kidney, heart, eye and brain. Through the reduction in glucose levels, an anti-thrombotic and anti-atherosclerotic effect can also be demonstrated. Reduction of glucose would prevent or minimize the glycosylation of any structural or functional protein within the cardio-renal system. This effect proves to be highly beneficial by evoking an additive or synergistic effect on vascular function/structure when administered with DPP-IV inhibitor which alone improves cardiovascular function and structure through a distinct mechanism.

Additionally, insulin resistance may contribute, in part, to the development of diabetes, hypertension and atherosclerosis (Fukuda et al., 2001). It is known that angiotensin II impairs insulin signaling (Fukuda et al., 2001) and that interruption of the renin angiotensin system with the use of an ACE inhibitor can partially restore insulin sensitivity (Sato et al., 1996; Nawano et al., 1999). Insulin can produce vasodilatation and lower blood pressure (Baron and Steinberg, 1996). The Zucker fatty rat, an animal model with insulin resistance, has been shown to possess a significantly higher blood pressure (Alonso-Galicia et al., 1996). ACE inhibition lowers blood pressure and improves insulin sensitivity in this model (Nawano et al., 1999). Combined administration of a cardiovascular compound as indicated in the present invention with a DPP-IV inhibitor will evoke further antihypertensive effects, improve vascular dynamics in hypertensive patients to a greater extent than after administration of either agent given alone. Interestingly, the co-administration of a cardiovascular compound and a DPP IV inhibitor will partially restore insulin sensitivity by preventing renin angiotensin system-induced impairment of insulin signaling pathways while at the same time raise insulin levels and improve glucose utilization. Consequently, combined administration will simultaneously improve both the metabolic and cardiovascular abnormalities, two conditions that often coexist in patients.

Further benefits are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

For example, it has turned out that the combination according to the present invention provides benefit especially in the treatment of modest hypertension or ISH that is beneficial to all diabetic patients regardless of their hypertensive status, e.g. reducing the risk of negative cardiovascular events by two different modes of action.

The DPP-IV inhibitor according to the present invention has proven to be useful in the treatment of type 2 diabetes mellitus and can likewise be used for the reduction of blood pressure in for example improving microalbuminuria. At sub-therapeutic doses, with respect to the treatment of hypertension, the combination according to the invention may be merely used for the treatment of diabetes, especially type 2 diabetes mellitus. In view of reduced dose of the DPP-IV inhibitor used according to the present invention, there is a considerable safety profile of the combination making it suitable for first line therapy.

Further benefits when applying the composition of the present invention are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

Preferably, the jointly therapeutically effective amounts of the active agents according to the combination of the present invention can be administered simultaneously or sequentially in any order, separately or in a fixed combination.

The pharmaceutical activities as effected by administration of the combination of active agents used according to the present invention can be demonstrated e.g. by using corresponding pharmacological models known in the pertinent art. The person skilled in the pertinent art is fully enabled to select a relevant animal test model to prove the hereinbefore and hereinafter indicated therapeutic indications and beneficial effects.

To evaluate the antihypertensive activity of the combination according to the invention, for example, the methodology as described by Lovenberg W: Animal models for hypertension research. Prog. Clin. Biol. Res. 1987, 229, 225-240 may be applied. For the evaluation that the combination according to the present invention may be used for the treatment of congestive heart failure, for example, the methods as disclosed by Smith H J, Nuttall A: Experimental models of heart failure. Cardiovasc Res 1985, 19, 181-186 may be applied. Molecular approaches such as transgenic methods are also described, for example by Luft et al.: Hypertension-induced end-organ damage. “A new transgenic approach for an old problem.” Hypertension 1999, 33, 212-218.

The insulin secretion enhancing properties of the combination according to the present invention may be determined by following the methodology as disclosed, for example, in the publication of T. Ikenoue et al. Biol. Pharm. Bull. 29(4), 354-359 (1997).

The corresponding subject matter of these references is herewith incorporated by reference in this specification.

The pharmaceutical composition according to the present invention as described herein before and hereinafter may be used for simultaneous use or sequential use in any order, for separate use or as a fixed combination.

Accordingly, the invention furthermore relates to a method for the prevention of, delay of progression of, treatment of a disease or condition selected from the group consisting of

(a) type 2 diabetes mellitus and related diseases, disorders or conditions (including but not limited to diabetic nephropathy, diabetic retinopathy and diabetic neuropathy);

(b) insulin resistance and syndrome X, obesity

(c) hypertension including hypertension in the elderly, familial dyslipidemic hypertension and isolated systolic hypertension (ISH); increased collagen formation, fibrosis, and remodeling following hypertension (antiproliferative effect of the combination); erectile dysfunction, impaired vascular compliance, stroke; all these diseases or conditions associated with or without hypertension,

(d) congestive heart failure, left ventricular hypertrophy, survival post myocardial infarction (MI), coronary artery diseases, atherosclerosis, angina pectoris, thrombosis,

(e) renal failure, especially chronic renal failure, glomerulosclerosis, nephropathy;

(f) hypothyroidism;

(g) endothelial dysfunction with or without hypertension,

(h) hyperlipidemia, hyperlipoproteinemia, hypertryglyceridemia, and hypercholesterolemia,

(i) macular degeneration, cataract, glaucoma,

(j) skin and connective tissue disorders, and

(k) restenosis after percutaneous transluminal angioplasty, and restenosis after coronary artery bypass surgery; peripheral vascular disease;

comprising administering to a warm-blooded animal, including man, in need thereof a jointly effective amount of a combination of a DPP IV inhibitor or a pharmaceutically acceptable salt thereof with at least one therapeutic agent selected from the group consisting of

(i) an AT₁-receptor antagonist or a pharmaceutically acceptable salt thereof,

(ii) an angiotensin converting enzyme (ACE) inhibitor or a pharmaceutically acceptable salt thereof,

(iii) a renin inhibitor or a pharmaceutically acceptable salt thereof,

(iv) a beta adrenergic receptor blocker or a pharmaceutically acceptable salt thereof,

(v) an alpha adrenergic receptor blocker or a pharmaceutically acceptable salt thereof,

(vi) a calcium channel blocker or a pharmaceutically acceptable salt thereof,

(vii) an aldosterone synthase inhibitor or a pharmaceutically acceptable salt thereof,

(viii) an aldosterone receptor antagonist or a pharmaceutically acceptable salt thereof,

(ix) a neutral endopeptidase (NEP) inhibitor or a pharmaceutically acceptable salt thereof,

(x) a dual angiotensin converting enzyme/neutral endopetidase (ACE/NEP) inhibitor or a pharmaceutically acceptable salt thereof,

(xi) an endothelin receptor antagonist or a pharmaceutically acceptable salt thereof, and

(xii) a diuretic or a pharmaceutically acceptable salt thereof.

Furthermore, the present invention relates to the use of a combination of a DPP IV inhibitor or a pharmaceutically acceptable salt thereof with at least one therapeutic agent selected from the group consisting of

(i) an AT₁-receptor antagonist or a pharmaceutically acceptable salt thereof,

(ii) an angiotensin converting enzyme (ACE) inhibitor or a pharmaceutically acceptable salt thereof,

(iii) a renin inhibitor or a pharmaceutically acceptable salt thereof,

(iv) a beta adrenergic receptor blocker or a pharmaceutically acceptable salt thereof,

(v) an alpha adrenergic receptor blocker or a pharmaceutically acceptable salt thereof,

(vi) a calcium channel blocker or a pharmaceutically acceptable salt thereof,

(vii) an aldosterone synthase inhibitor or a pharmaceutically acceptable salt thereof,

(viii) an aldosterone receptor antagonist or a pharmaceutically acceptable salt thereof,

(ix) a neutral endopeptidase (NEP) inhibitor or a pharmaceutically acceptable salt thereof,

(x) a dual angiotensin converting enzyme/neutral endopetidase (ACE/NEP) inhibitor or a pharmaceutically acceptable salt thereof,

(xi) an endothelin receptor antagonist or a pharmaceutically acceptable salt thereof, and

(xii) a diuretic or a pharmaceutically acceptable salt thereof;

for the manufacture of a medicament for the prevention of, delay of progression of, or treatment of a disease or condition selected from the group consisting of

(a) type 2 diabetes mellitus and related diseases, disorders or conditions (including but not limited to diabetic nephropathy, diabetic retinopathy and diabetic neuropathy);

(b) insulin resistance and syndrome X, obesity

(c) hypertension including hypertension in the elderly, familial dyslipidemic hypertension, and isolated systolic hypertension (ISH); increased collagen formation, fibrosis, and remodeling following hypertension (antiproliferative effect of the combination); erectile dysfunction, impaired vascular compliance, stroke; all these diseases or conditions associated with or without hypertension,

(d) congestive heart failure, left ventricular hypertrophy, survival post myocardial infarction (MI), coronary artery diseases, atherosclerosis, angina pectoris, thrombosis,

(e) renal failure, especially chronic renal failure, glomerulosclerosis, nephropathy;

(f) hypothyroidism;

(g) endothelial dysfunction with or without hypertension,

(h) hyperlipidemia, hyperlipoproteinemia, hypertryglyceridemia, and hypercholesterolemia,

(i) macular degeneration, cataract, glaucoma,

(j) skin and connective tissue disorders, and

(k) restenosis after percutaneous transluminal angioplasty, and restenosis after coronary artery bypass surgery; peripheral vascular disease;

The invention furthermore relates to a pharmaceutical composition for the prevention of, delay of progression of, treatment of a disease or condition selected from the group consisting of

(a) type 2 diabetes mellitus and related diseases, disorders or conditions (including but not limited to diabetic nephropathy, diabetic retinopathy and diabetic neuropathy);

(b) insulin resistance and syndrome X, obesity;

(c) hypertension including hypertension in the elderly, familial dyslipidemic hypertension, and isolated systolic hypertension (ISH); increased collagen formation, fibrosis, and remodeling following hypertension (antiproliferative effect of the combination); erectile dysfunction, impaired vascular compliance, stroke; all these diseases or conditions associated with or without hypertension;

(d) congestive heart failure, left ventricular hypertrophy, survival post myocardial infarction (MI), coronary artery diseases, atherosclerosis, angina pectoris, thrombosis;

(e) renal failure, especially chronic renal failure, glomerulosclerosis, nephropathy;

(f) hypothyroidism;

(g) endothelial dysfunction with or without hypertension;

(h) hyperlipidemia, hyperlipoproteinemia, hypertryglyceridemia, and hypercholesterolemia;

(i) macular degeneration, cataract, glaucoma;

(j) skin and connective tissue disorders, and

(k) restenosis after percutaneous transluminal angioplasty, and restenosis after coronary artery bypass surgery; peripheral vascular disease;

comprising a combination of a DPP IV inhibitor or a pharmaceutically acceptable salt thereof with at least one therapeutic agent selected from the group consisting of

(i) an AT1-receptor antagonist or a pharmaceutically acceptable salt thereof,

(ii) an angiotensin converting enzyme (ACE) inhibitor or a pharmaceutically acceptable salt thereof,

(iii) a renin inhibitor or a pharmaceutically acceptable salt thereof,

(iv) a beta adrenergic receptor blocker or a pharmaceutically acceptable salt thereof,

(v) an alpha adrenergic receptor blocker or a pharmaceutically acceptable salt thereof,

(vi) a calcium channel blocker or a pharmaceutically acceptable salt thereof,

(vii) an aldosterone synthase inhibitor or a pharmaceutically acceptable salt thereof,

(viii) an aldosterone antagonist or a pharmaceutically acceptable salt thereof,

(ix) a neutral endopeptidase (NEP) inhibitor or a pharmaceutically acceptable salt thereof,

(x) a dual angiotensin converting enzyme/neutral endopetidase (ACE/NEP) inhibitor or a pharmaceutically acceptable salt thereof,

(xi) an endothelin receptor antagonist or a pharmaceutically acceptable salt thereof, and

(xii) a diuretic or a pharmaceutically acceptable salt thereof;

and a pharmaceutically acceptable carrier.

Preferably, the jointly therapeutically effective amounts of the active agents according to the combination of the present invention can be administered simultaneously or sequentially in any order, separately or in a fixed combination.

The pharmaceutical composition according to the present invention as described hereinbefore and hereinafter may be used for simultaneous use or sequential use in any order, for separate use or as a fixed combination.

A further aspect of the present invention is a kit for the prevention of, delay of progression of, treatment of a disease or condition according to the present invention comprising

(a) an amount of a DPP IV inhibitor or a pharmaceutically acceptable salt thereof in a first unit dosage form;

(b) an amount of at least one therapeutic agent selected from the group consisting of components (i) to (xii), or, in each case, where appropriate, a pharmaceutically acceptable salt thereof in a second etc. unit dosage form; and

(c) a container for containing said first, second etc. unit forms.

In a variation thereof, the present invention likewise relates to a “kit-of-parts”, for example, in the sense that the components to be combined according to the present invention can be dosed independently or by use of different fixed combinations with distinguished amounts of the components, i.e. simultaneously or at different time points. The parts of the kit of parts can then e.g. be administered simultaneously or chronologically staggered, that is at different time points and with equal or different time intervals for any part of the kit of parts. Preferably, the time intervals are chosen such that the effect on the treated disease or condition in the combined use of the parts is larger than the effect that would be obtained by use of only any one of the components.

The present invention thus also relates to a kit of parts comprising

(a) an amount of a DPP IV inhibitor or a pharmaceutically acceptable salt thereof in a first unit dosage form;

(b) an amount of at least one therapeutic agent selected from the group consisting of components (i) to (xii), or, in each case, where appropriate, a pharmaceutically acceptable salt thereof, in the form of two or three or more separate units of the components (i) to (xii).

The invention furthermore relates to a commercial package comprising the combination according to the present invention together with instructions for simultaneous, separate or sequential use.

In a preferred embodiment, the (commercial) product is a commercial package comprising as active ingredients the combination according to the present invention (in the form of two or three or more separate units of the components (i) to (xii)), together with instructions for its simultaneous, separate or sequential use, or any combination thereof, in the delay of progression or treatment of the diseases (a) to (k) as mentioned herein.

All the preferences mentioned herein apply to the combination, composition, use, method of treatment, “kit of parts” and commercial package of the invention.

These pharmaceutical preparations are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compound. Pharmaceutical preparations for enteral or parenteral, and also for ocular, administration are, for example, in unit dose forms, such as coated tablets, tablets, capsules or suppositories and also ampoules. These are prepared in a manner that is known per se, for example using conventional mixing, granulation, coating, solubilizing or lyophilizing processes. Thus, pharmaceutical preparations for oral use can be obtained by combining the active compound with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

Preferred dosages for the active ingredients of the pharmaceutical combination according to the present invention are therapeutically effective dosages, especially those which are commercially available.

Normally, in the case of oral administration, an approximate daily dose of from about 1 mg to about 360 mg is to be estimated e.g. for a patient of approximately 75 kg in weight.

The dosage of the active compound can depend on a variety of factors, such as mode of administration, homeothermic species, age and/or individual condition.

The pharmaceutical preparation will be supplied in the form of suitable dosage unit form, for example, a capsule or tablet, and comprising an amount, being together with the further component(s) jointly effective, e.g.

The doses of DPP-IV inhibitor of formula (I) to be administered to warm-blooded animals, for example human beings, of, for example, approximately 70 kg body weight, especially the doses effective in the inhibition of the enzyme renin, e.g. in lowering blood pressure and/or in improving the symptoms of glaucoma, are from approximately 3 mg to approximately 3 g, preferably from approximately 10 mg to approximately 1 g, for example approximately from 20 mg to 200 mg, per person per day, divided preferably into 1 to 4 single doses which may, for example, be of the same size. Usually, children receive about half of the adult dose. The dose necessary for each individual can be monitored, for example by measuring the serum concentration of the active ingredient, and adjusted to an optimum level. Single doses comprise, for example, 10, 40 or 100 mg per adult patient.

Valsartan, as a representative of the class of AT₁-receptor antagonists, will be supplied in the form of suitable dosage unit form, for example, a capsule or tablet, and comprising a therapeutically effective amount, e.g. from about 20 to about 320 mg, of valsartan which may be applied to patients. The application of the active ingredient may occur up to three times a day, starting e.g. with a daily dose of 20 mg or 40 mg of valsartan, increasing via 80 mg daily and further to 160 mg daily up to 320 mg daily. Preferably, valsartan is applied twice a day with a dose of 80 mg or 160 mg, respectively, each. Corresponding doses may be taken, for example, in the morning, at mid-day or in the evening.

Preferred dosage unit forms of ACE inhibitors are, for example, tablets or capsules comprising e.g. from about 5 mg to about 40 mg, preferably 5 mg, 10 mg, 20 mg or 40 mg, of benazepril; from about 6.5 mg to 100 mg, preferably 6.25 mg, 12.5 mg, 25 mg, 50 mg, 75 mg or 100 mg, of captopril; from about 2.5 mg to about 40 mg, preferably 2.5 mg, 5 mg, 10 mg, 20 mg or 40 mg, of enalapril; from about 11 mg to about 40 mg, preferably 10 mg or 20 mg, of fosinopril; from about 2 mg to about 8 mg, preferably 2 mg or 4 mg, of perindopril; from about 5 mg to about 40 mg, preferably 5 mg, 10 mg or 20 mg, of quinapril; or from about 1.25 mg to about 20 mg, preferably 1.25 mg, 2.5 mg, or 5 mg, of ramipril.

Preferred dosage unit forms of renin inhibitors are, for example, tablets or capsules comprising e.g. from about 5 mg to about 500 mg, preferably, when using aliskiren, for example, 50 to 250 mg (equivalent to the free acid) of aliskiren, for example, administered once a day.

Preferred dosage unit forms of beta blockers are, for example, tablets or capsules comprising e.g. from about 25 mg to 100 mg, especially 25 mg, 50 mg or 100 mg, of atenolol; from about 5 2.5 to 10 mg, especially 2.5 mg, 5 mg or 10 mg, of bisoprolol, especially the fumarate thereof; from about 50 to 200 mg, especially 50 mg, 100 mg or 200 mg, of metoprolol, especially the hemi-(R,R)fumarate or the fumarate thereof; from about 100 mg to 2.5 g, especially 100 mg or 2.5 g, of esmolol especially the hydrochloride thereof; 200 mg of celiprolol, especially the hydrochloride thereof; from about 50 mg to 100 mg, especially 60 mg or 100 mg, of talinolol; from about 200 my to 800 mg, especially 200 mg or 400 mg, of acebutolol, especially the hydrochloride thereof; from about 10 mg to 30 mg, especially 10 my or 20 mg, of timolol, especially the maleate thereof; from about 5 mg to 20 mg, especially 5 mg, 10 mg, or 20 mg of betaxolol, especially the hydrochloride thereof; from about 20 mg to 80 mg, especially 20 mg, 40 mg, or 80 mg of nadolol, from about 40 mg to 160 mg, especially, 40 mg, 80 my or 160 mg, of oxprenolol, especially the hydrochloride thereof; from about 5 mg to 40 mg, especially, 5 mg, 10 mg, 20 mg or 40 mg, of pindolol; from about 25 mg to 160 mg, especially 25 mg, 40 mg, 80 mg, 100 mg or 160 mg, of propranolol, especially the hydrochloride thereof; from about 50 mg to 100 mg, especially 50 mg or 100 mg, of bupranolol, especially the hydrochloride thereof; from about 2.5 to 40 mg, especially 2.5 mg, 5 mg, 10 mg, 20 mg, or 40 mg of penbutolol, especially the sulphate thereof; from about 2.5 my to 10 mg, especially 2.5 mg, 5 mg or 10 mg, of carteolol, especially the hydrochloride thereof; from about 3.125 mg to 25 mg, especially 3.125 mg, 6.25 mg, 12.5 mg or 25 mg of carvedilol, from about 100 mg to 800 mg, especially 100 mg, 200 mg, 400 mg or 800 mg of labetalol, especially the hydrochloride thereof.

Preferably, in case of free combinations, preferred are those dosages for launched products that have been approved and that have been marketed.

Especially preferred are low dose combinations.

What is claimed is:

1. A pharmaceutical composition, comprising:
a DPP IV inhibitor or a pharmaceutically acceptable salt thereof and
a cardiovascular compound, being different from a statin, or a pharmaceutically acceptable salt thereof.

2. The pharmaceutical composition of claim 1, comprising:
a DPP IV inhibitor or a pharmaceutically acceptable salt thereof and at least one therapeutic agent selected from the group consisting of
(i) an AT₁-receptor antagonist or a pharmaceutically acceptable salt thereof
(ii) an angiotensin converting enzyme (ACE) inhibitor or a pharmaceutically acceptable salt thereof,
(iii) a renin inhibitor or a pharmaceutically acceptable salt thereof,
(iv) a beta adrenergic receptor blocker or a pharmaceutically acceptable salt thereof,
(v) an alpha adrenergic receptor blocker or a pharmaceutically acceptable salt thereof
(vi) a calcium channel blocker or a pharmaceutically acceptable salt thereof,
(vii) an aldosterone synthase inhibitor or a pharmaceutically acceptable salt thereof,
(viii) an aldosterone receptor antagonist or a pharmaceutically acceptable salt thereof,
(ix) a neutral endopeptidase (NEP) inhibitor or a pharmaceutically acceptable salt thereof,
(x) a dual angiotensin converting enzyme/neutral endopetidase (ACE/NEP) inhibitor or a pharmaceutically acceptable salt thereof
(xi) an endothelin receptor angonist or a pharmaceutically acceptable salt thereof, and
(xii) a diuretic or a pharmaceutically acceptable salt thereof.

3. The pharmaceutical composition of claim 1 wherein the DPP-IV inhibitor is (S)-1-{2-[5-cyanopyridin-2-yl)amino]ethyl-aminoacetyl)-2-cyano-pyrrolidine or (S)-1-[(3-hydroxy-1-adamantyl)amino]acetyl-2-cyano-pyrrolidine.

4. The pharmaceutical composition of claim 2, wherein the AT₁-receptor antagonist is losartan, olmesartan or valsartan;
ACE inhibitor is benazepril, enalapril, lisinopril or ramipril;
renin inhibitor is aliskiren;
beta blocker is metoprolol;
alpha blocker is doxazosin
calcium channel blocker is amlodipine;
aldosterone synthase inhibitor is fadrozole or (+)-enantiomer of fadrozole;
aldosterone receptor antagonist is eplerenone;
neutral endopeptidase inhibitor is candoxatril or sinorphan
dual angiotensin converting enzyme/neutral endopetidase (ACE/NEP) inhibitor is omapatrilat;
endothelin receptor antagonist is bosentan;
diuretic is hydrochlorothiazide
or, in each case, a pharmaceutically acceptable salt thereof.

5. The pharmaceutical composition of claim 1, comprising (S)-1-{2-[5-cyanopyridin-2-yl)amino]ethyl-aminoacetyl)-2-cyano-pyrrolidine or (S)-1-[(3-hydroxy-1-adamantyl)amino]acetyl-2-cyano-pyrrolidine or a pharmaceutically acceptable salt thereof and valsartan or a pharmaceutically acceptable salt thereof or aliskiren or a pharmaceutically acceptable salt thereof.

6. A method for the treatment of a disease or condition selected from the group consisting of
(a) type 2 diabetes mellitus and related diseases, disorders or conditions;
(b) insulin resistance and syndrome X, obesity;
(c) hypertension including hypertension in the elderly, familial dyslipidemic hypertension, and isolated systolic hypertension (ISH); increased collagen formation, fibrosis, and remodeling following hypertension; erectile dysfunction, impaired vascular compliance, stroke; all these diseases or conditions associated with or without hypertension;
(d) congestive heart failure, left ventricular hypertrophy, survival post myocardial infarction (MI), coronary artery diseases, atherosclerosis, angina pectoris, thrombosis;
(e) renal failure, especially chronic renal failure, glomerulosclerosis, nephropathy;
(f) hypothyroidism;
(g) endothelial dysfunction with or without hypertension;
(h) hyperlipidemia, hyperlipoproteinemia, hypertryglyceridemia, and hypercholesterolemia;
(i) macular degeneration, cataract, glaucoma;
(j) skin and connective tissue disorders, and
(k) restenosis after percutaneous transluminal angioplasty, and restenosis after coronary artery bypass surgery; peripheral vascular disease;
comprising:
administering to a warm-blooded animal in need thereof a jointly effective amount of a combination of a DPP IV inhibitor or a pharmaceutically acceptable salt thereof with at least one therapeutic agent selected from the group consisting of
(i) an AT₁-receptor antagonist or a pharmaceutically acceptable salt thereof,
(ii) an angiotensin converting enzyme (ACE) inhibitor or a pharmaceutically acceptable salt thereof,
(iii) a renin inhibitor or a pharmaceutically acceptable salt thereof,
(iv) a beta adrenergic receptor blocker or a pharmaceutically acceptable salt thereof,
(v) an alpha adrenergic receptor blocker or a pharmaceutically acceptable salt thereof,
(vi) a calcium channel blocker or a pharmaceutically acceptable salt thereof,
(vii) an aldosterone synthase inhibitor or a pharmaceutically acceptable salt thereof,
(viii) an aldosterone receptor antagonist or a pharmaceutically acceptable salt thereof,
(ix) a neutral endopeptidase (NEP) inhibitor or a pharmaceutically acceptable salt thereof,
(x) a dual angiotensin converting enzyme/neutral endopetidase (ACE/NEP) inhibitor or a pharmaceutically acceptable salt thereof,
(xi) an endothelin receptor antagonist or a pharmaceutically acceptable salt thereof, and
(xii) a diuretic or a pharmaceutically acceptable salt thereof.

7. (canceled)

8. A kit of parts comprising
(a) an amount of a DPP IV inhibitor or a pharmaceutically acceptable salt thereof in a first unit dosage form;
(b) an amount of at least one therapeutic agent selected from the group consisting of
(i) an AT₁-receptor antagonist or a pharmaceutically acceptable salt thereof,
(ii) an angiotensin converting enzyme (ACE) inhibitor or a pharmaceutically acceptable salt thereof,
(iii) a renin inhibitor or a pharmaceutically acceptable salt thereof,
(iv) a beta adrenergic receptor blocker or a pharmaceutically acceptable salt thereof,
(v) an alpha adrenergic receptor blocker or a pharmaceutically acceptable salt thereof,
(vi) a calcium channel blocker or a pharmaceutically acceptable salt thereof
(vii) an aldosterone synthase inhibitor or a pharmaceutically acceptable salt thereof,
(viii) an aldosterone receptor antagonist or a pharmaceutically acceptable salt thereof,
(ix) a neutral endopeptidase (NEP) inhibitor or a pharmaceutically acceptable salt thereof,
(x) a dual angiotensin converting enzyme/neutral endopetidase (ACE/NEP) inhibitor or a pharmaceutically acceptable salt thereof,
(xi) an endothelin receptor antagonist or a pharmaceutically acceptable salt thereof, and
(xii) a diuretic or,
in each case, where appropriate, a pharmaceutically acceptable salt thereof, in the form of two or three or more separate units of the components (i) to (xii).

9. The method of claim 6, wherein the
DPP-IV inhibitor is (S)-1-{2-[5-cyanopyridin-2-yl)amino]ethyl-aminoacetyl)-2-cyano-pyrrolidine or (S)-1-[(3-hydroxy-1-adamantyl)amino]acetyl-2-cyano-pyrrolidine, and wherein the AT₁-receptor antagonist is losartan, olmesartan or valsartan;
ACE inhibitor is benazepril, enalapril, lisinopril or ramipril;
renin inhibitor is aliskiren;
beta blocker is metoprolol;
alpha blocker is doxazosin
calcium channel blocker is amlodipine;
aldosterone synthase inhibitor is fadrozole or (+)-enantiomer of fadrozole;
aldosterone receptor antagonist is eplerenone;
neutral endopeptidase inhibitor is candoxatril or sinorphan
dual angiotensin converting enzyme/neutral endopetidase (ACE/NEP) inhibitor is omapatrilat;
endothelin receptor antagonist is bosentan;
diuretic is hydrochlorothiazide
or, in each case, a pharmaceutically acceptable salt thereof.

10. The pharmaceutical composition of claim 2, comprising (S)-1-{2-[5-cyanopyridin-2-yl)amino]ethyl-aminoacetyl)-2-cyano-pyrrolidine or (S)-1-[(3-hydroxy-1-adamantyl)amino]acetyl-2-cyano-pyrrolidine or a pharmaceutically acceptable salt thereof and valsartan or a pharmaceutically acceptable salt thereof or aliskiren or a pharmaceutically acceptable salt thereof.

11. The method of treatment according to claim 6, wherein the warm blooded animal is a human.
